(19) United States
(12) Patent Application Publication
(10) Pub. No.: US 2020/0390366 A1
(43) Pub. Date: Dec. 17, 2020

(54) Apparatus And System For Measuring One Or More Parameters Of A Person's Foot

(71) Applicant: Shoes2Run Limited, Washington Tyne and Wear (GB)

(72) Inventors: Craig Downs, Washington Tyne and Wear (GB); Martin Coleman, Washington Tyne and Wear (GB)

(21) Appl. No.: 16/612,148

(22) PCT Filed: May 14, 2018
(86) PCT No.: PCT/GB2018/051291

§ 371 Date: Nov. 8, 2019

(30) Foreign Application Priority Data
May 12, 2017 (GB) 1707619.1

(51) Int. Cl. A61B 5/103; A61B 5/00; A43B 3/00; A43D 1/02; G01P 15/18; G01C 19/00

(52) U.S. Cl. CPC A61B 5/1038; A61B 5/6829; A61B 5/0002; A61B 5/4884; A61B 5/7475; A61B 2562/0247; A43D 1/027; G01P 15/18; G01C 19/00; A61B 2562/0219; A43B 3/0005

(57) ABSTRACT

A system, device and method for generating date representative of a person and using that data as criteria in a selection made for that person. The system includes a device worn on or adjacent to a user's foot. The device includes sensing means with an accelerometer and/or gyroscope and/or pressure sensing means to measure parameters of a person's foot during physical activities. The parameter data is collected and transmitted to a data processing means to generate a representative data group for the person. Matching means compare the representative data group to data stored in memory. Group data includes data representative of a range of footwear. Indication means are controlled to indicate, from the result of the matching means, any of the range of footwear which are selected.

The invention to which this application relates is apparatus and a system which allows the determination of the characteristic of at least one parameter, more typically, a number of parameters, of a person's foot and the parameters can then be used to determine a particular footwear item which is best suited for that person.

Conventionally, when a person goes to buy a new pair of shoes, then, regardless of the particular use of the shoes, they can request that the retail outlet measure the size of their foot which can be from heel to toe and/or measure the width of the person's foot. Most often the item of footwear is then chosen. However, when the item of footwear is to be worn for a sports activity and, in particular, an activity which involves the footwear being worn for a significant period of time such as by a jogger or competitive runner, it is typically the case that more parameter measurements are required to be taken in order to ensure that the item of footwear which is chosen matches a person's running style and reduces the risk of causing an injury to the wearer. These further parameters can be measured in their most basic form, by a person wearing their normal pair of running shoes outside of a shop and the person who is selling the shoes viewing the running action. Alternatively the running action may be recorded and the running action of the person subsequently analysed.

Increasingly, a more sophisticated process is available in specialist running footwear shops and which involves the person who wishes to purchase the shoes running on a treadmill within the retail outlet and certain parameters of the running action and/or person's feet being measured through gait analysis. This process is also available via a qualified podiatrist and/or sport physiotherapist.

While this is undoubtedly preferable, there are still problems, not least being the retail space which is required in order to accommodate the treadmill and associated apparatus and, in many retail outlets, this is not possible. Furthermore, the equipment is relatively expensive and therefore it tends to be that only the largest footwear retail outlets are provided with this apparatus and/or the apparatus is provided to be used in conjunction with the sale of a particular brand of shoes.

Furthermore, in relatively busy times queues can start to form for potential customers wishing to use the apparatus and, if they are not able to use the apparatus or have to wait a relatively long time to do so, then they may feel that have been given an inferior service and they may decide not to purchase footwear from that retail outlet. Furthermore, the apparatus and systems which are conventionally available are required to be used at a retail outlet which means that the same cannot be used and the available data utilised for purchases which are made over the internet.

It is also known to provide relatively complex devices that will provide data indicative of a runner's performance, these devices are provided to generate a historical record of the running performance rather than to allow any analysis of the shoes worn or which should be purchased.

Thus, while it is appreciated that there is a need to provide additional foot parameter measurements in order to allow items of footwear which are best suited to a person's foot to be chosen, the current systems and apparatus which are available, have significant disadvantages and so it is found that in practice, the majority of footwear, even footwear for runners, is still purchased without any significant parameter data being available with respect to the person's feet.

These disadvantages can lead to the wearer purchasing a pair of shoes which are not suitable for their feet or running style and which can cause discomfort or, in a worst case scenario, can cause long term injuries to the wearer.

In a first aspect of the invention there is provided a system for generating data representative of a person and using that data as criteria in a selection made for that person, said system including a device to be worn on or adjacent to a foot of the user, wherein said device includes sensing means including an accelerometer and/or a gyroscope and/or a pressure sensing means to allow one or more parameters relating to that person's foot to be measured during one or more physical activities performed by the person and data relating to the one or more parameters is collected and transmitted via a transmission means to a data processing means to allow a representative data group to be generated for the said person and matching means compare the said representative data group to data stored in a memory which includes data representative of a range of footwear and indication means are controlled to indicate, from the result of the matching means, any of die said range of footwear which are selected to be suggested to be worn by the said user.

In one embodiment the sensing means is attached to the foot via a carrier.

In one embodiment the communication of the data relating to the parameters is transferred from the device via a portable communication device such as a smart phone application which communicates in one embodiment, with the sensing means utilising wireless Bluetooth communications technology and transmits the data using wireless transmission to the data processing means.

In one embodiment a first data processing means is provided which includes a pre-processing SQL database server which collects the relayed consumer data from the smart phone via the smart phone's internet data connection and which includes an AI algorithm which presents the data to a post processing database.

Typically the AI algorithm analyses and processes the consumer data, which outputs the final consumer result to a second SQL database which receives the final AI consumer data output result and matches the result to a memory in which data relating to an available range of shoes is held and which matches the representative data group with the shoe description data to recommend the most suitable type or types of shoe(s) available in the database.

In one embodiment the person is able to further refine the recommendation based on their personal preferences.

In one embodiment the processing means allows a recommendation of one or more items of footwear to be made for the said person based on the measured parameter data.

In one embodiment the processing means refers to data relating to the range of footwear to compare the measured data with the same when determining which items of footwear are suitable.

In one embodiment, the device can be worn at any location and therefore is not required to be provided in situ at a particular location such as a retail outlet.

In one embodiment, the data which is generated by the device in relation to the one or more parameters, can be stored and/or communicated to a remote location, such as a cloud database and at which location the cloud database will process the data with reference to the range of available items of footwear on the market at that moment in time and, if available, recommend one or more items of footwear which are best suited to the person's feet as represented by the data.

In one embodiment the person can enter any retail store and via a smartphone app on their mobile phone, enter a specific manufacturer and model of a shoe and the app will be able to inform them if that particular shoe is right for them based on a comparison of the parameter measurements for that person which have previously been obtained from the device and the predefined data which the app has, or can access, in relation to characteristics of that particular shoe which has been selected in accordance with the invention.

Typically, the parameter measurements are identified for both feet and data is provided for both feet when deciding on the best pair of shoes to be worn.

In one embodiment there may be provided a link to allow the person when presented with the suggestion of footwear which is suited to them, to make a purchase of one or more pairs of said footwear via a link to one or more retailers of the footwear.

In a further aspect of the invention there is provided a system for generating data representative of a person and using that data as criteria in a selection made for that person, said system including a device to be worn on or adjacent to a foot of the user, wherein said device includes sensing means to allow one or more parameters relating to that person to be measured during one or more physical activities performed by the person and data relating to the one or more parameters is collected and transmitted via a transmission means to a data processing means to allow a representative data group to be generated for the said person and matching means compare the said representative data group to data stored in a memory and indication means are controlled to indicate the result of the matching means.

In one embodiment the sensing means include an accelerometer and/or a gyroscope and/or a pressure sensing means.

In one embodiment the at least one parameter is linked to the person's foot.

In one embodiment the said data stored in memory with which the representative data group is compared is data relating to a range of footwear which is available to be purchased.

In one embodiment the said data stored in memory with which the representative data group is compared is historical data relating to one or more elements relating to that persons health.

In one embodiment the said data stored in memory with which the representative data group is compared is historical data relating to one or more elements relating to expected health values.

In one embodiment in addition to the data generated from the said device, further data relating to other parameters are input and used in the processing of the data for that person.

In one embodiment the said further data includes any or any combination of height, weight and/or age of the person and/or type of terrain in which footwear is to be worn and/or other criteria relating to the footwear such as price and/or colour.

In a further aspect of the invention there is provided a device to be mounted in position on or adjacent to a foot of a person, wherein said device includes sensing means including an accelerometer and/or a gyroscope and/or a pressure sensor to allow one or more parameters relating to that person's foot to be measured during one or more physical activities performed by the person and data relating to the one or more parameters is collected and transmitted via a transmission means from the device for subsequent processing.

In one embodiment the device is provided to be located when in use in a carrier which includes engagement means, or is formed in a manner, so as to allow engagement of the carrier with the foot and to maintain the device in a required position with respect to the foot during the performance of the one or more activities.

Typically, the carrier is in the form of a sock or part of a sock, a bracelet, anklet or the like which fits around the ankle of the wearer and/or round the heel of the wearer so as to be located to provide the required data protection points on the wearer's foot. In one embodiment the device will attach to a person's foot or ankle and collect measurement data linked to a plurality of biodynamic parameters of the foot when the customer is asked to perform a short running exercise.

In one embodiment the apparatus comprises a wearable sensor device, a smart phone application and a cloud resident database.

In a further aspect of the invention there is provided a method for the analysis of one or more parameters relating to a person's foot, said method comprising the steps of providing and using a combination of sensing means provided as part of a device and locating the device on or adjacent to the foot whilst the person performs at least a walking and/or running action for a period of time and/or for a particular distance, collecting data from the sensing means which is created during the movement of the person's foot, transmitting the data from the device to data processing means and processing the data using one or a series of algorithms to output a representative data group related to that person to a post processing database and performing a matching exercise with respect to a database of data relating to a range of shoes and upon the results of the match selecting one or more pairs of shoes from the range which are suggested to the person as being best suited for them to wear.

Typically each of the available pairs of shoes are allocated specific identity data so as to allow the same to be matched, or not, with the representative data group for a person.

In one embodiment the matched shoe type recommendations are presented to the consumer via a user-friendly GUI dashboard interface which can be viewed on a smart phone or within a PC/Mac web browser interface.

In one embodiment the initial data is collected from the device via a Bluetooth connection, to a transmitting device, such as a smart phone, which then transmits the collected data to a data processing means at which the algorithms are processed. Typically the data processing means and/or post processing means are a facility which is remote from the use of the device and in one embodiment is capable of receiving and process g collected data from a number of devices.

Specific embodiments of the invention are now described with reference to the accompanying drawings wherein

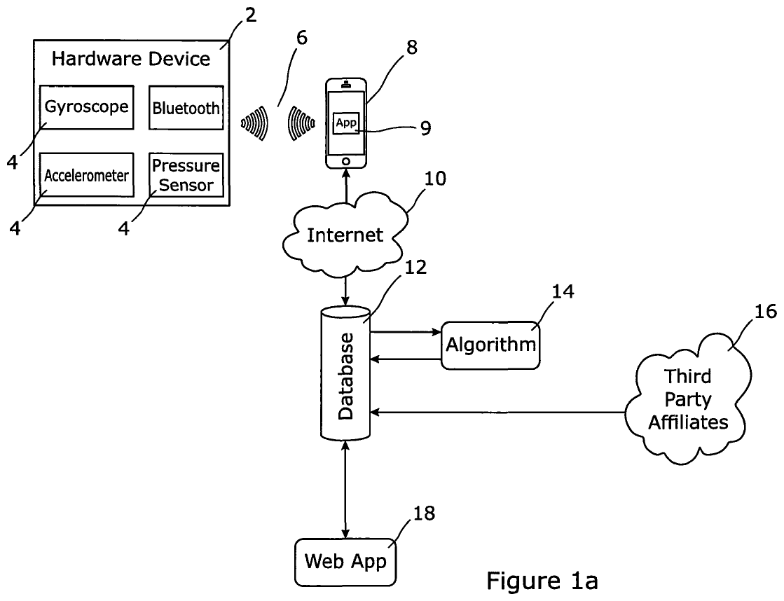

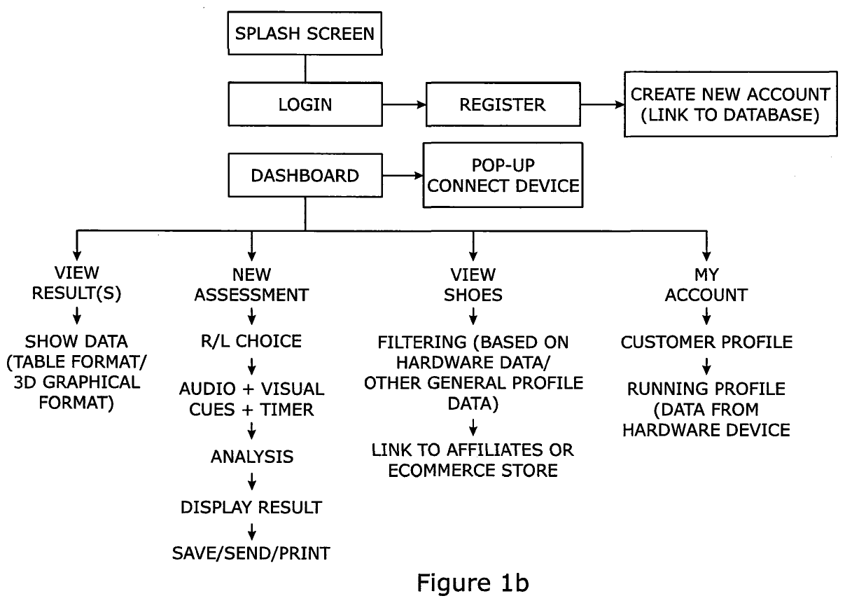

FIGS. 1a and b illustrate schematically, a system in accordance with one embodiment of the invention;

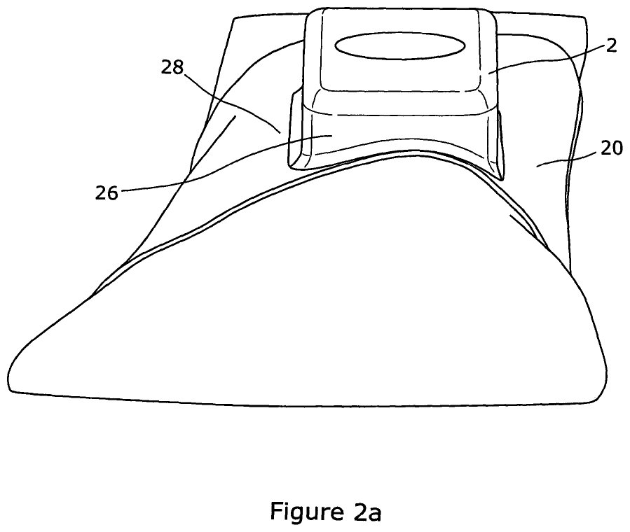

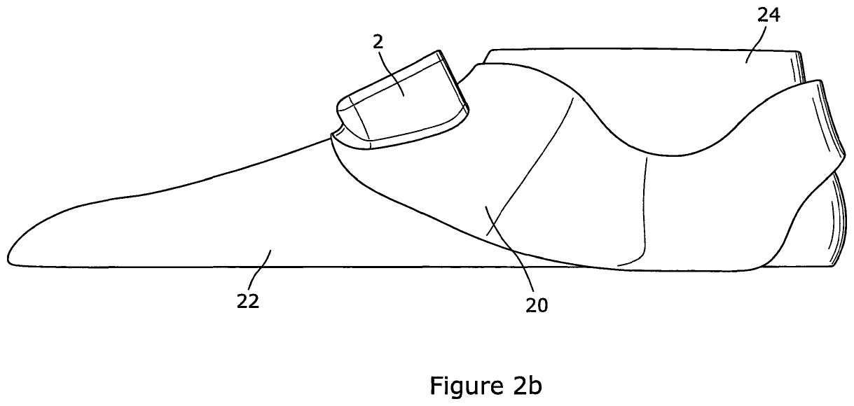

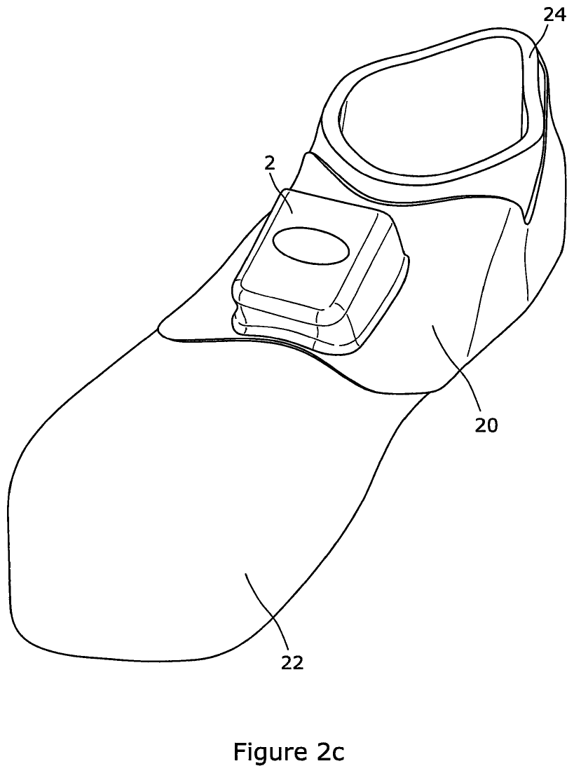

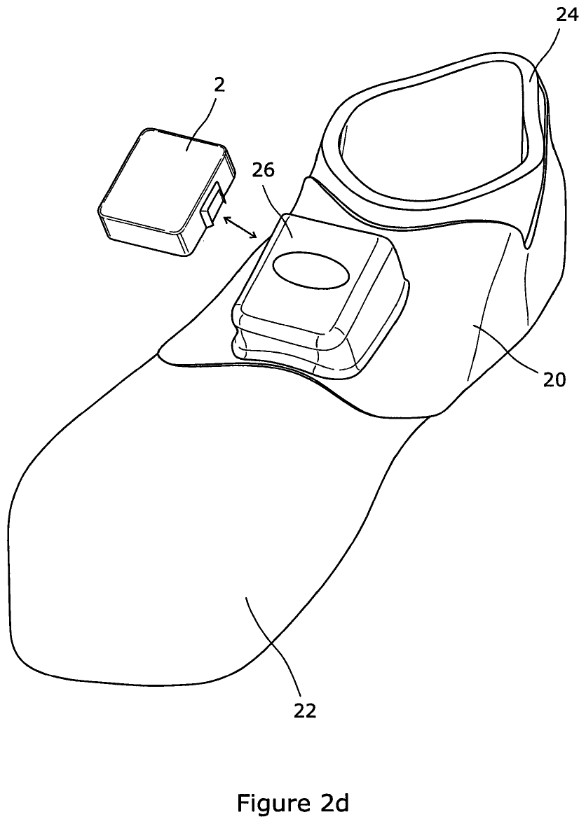

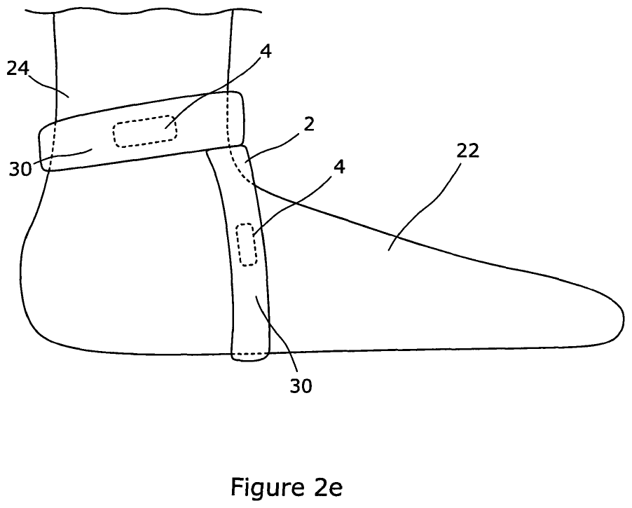

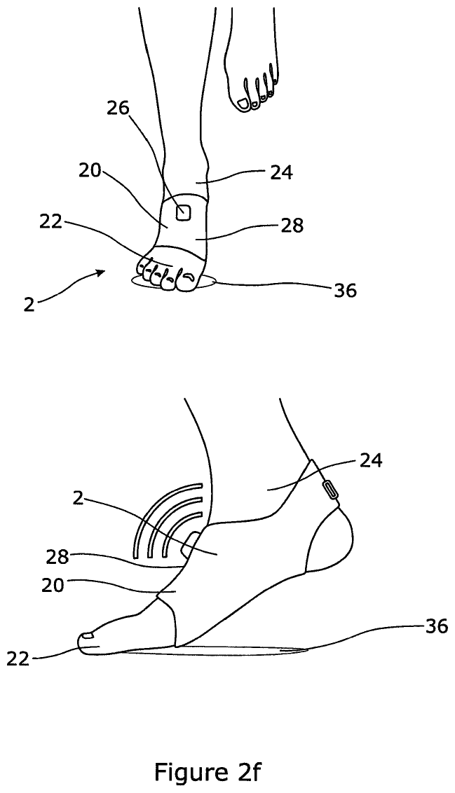

FIGS. 2a-f illustrate embodiments of the device and support means for positioning the same on the foot of a person in accordance with the invention; and

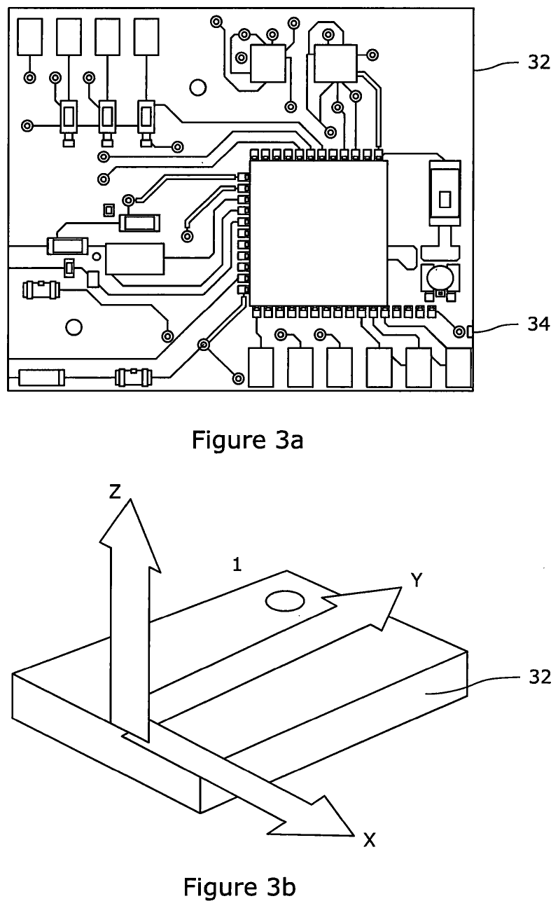

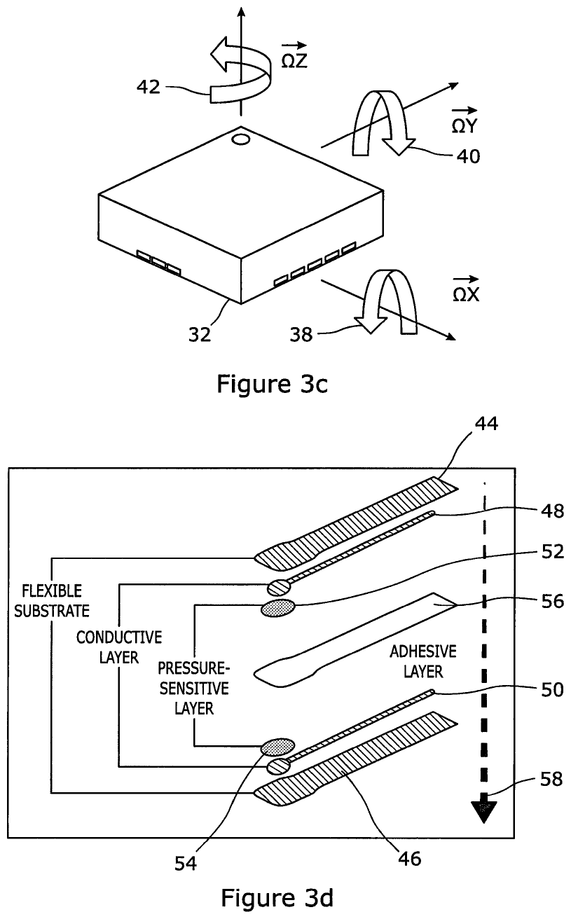

FIGS. 3a-d illustrate one embodiment of the components of the device in accordance with the invention.

Referring firstly to FIG. 1a, there is illustrated, schematically, a system in accordance with one embodiment of the invention. The system includes a device 2 which will be described subsequently and the device is provided with one or more sensors 4, 4′, 4″ thereon, said sensors provided of a type and/or at a location so as to allow data for specific parameters of a person's foot on which the device is worn, to be detected.

Typically, the parameters which are detected are selected by the provider of the device and, in one embodiment may be selected with regard to particular parameters which are relevant to the type of footwear which is to be chosen by the person using the device as a result of the data and/or with regard to the particular sport or activity which is to be performed by the person when wearing the footwear which is to be purchased.

Yet further, different weighting levels can be provided to the data from the various detected parameters in relation to the type of footwear and/or activity such that, for example, a parameter which is particularly important with regard to the foot when a person is playing basketball, may be less important when the item of footwear is to be worn by a person who is taking part in a marathon.

It will therefore be appreciated that the device and system can be controlled so as to provide selected characteristics in relation to the particular use to which the same is to be used at that time.

The device is provided in two way communication with a user interface and data collecting means which in this embodiment are provided as an application 9 on a smartphone 8. The collected data is then transmitted from the smartphone, typically using the internet and cloud storage 10, to data processing means 12 at which the collected data is processed in accordance with predetermined algorithms 14 to generate a group of data which is representative of parameters of the person who is wearing the device at that time. Data from third parties 16 such as shoe manufacturers is provided which is representative of the shoe types which are available and further data processing attempts to find one or more matches between the group of representative data and the data for the shoe types and, if matches are found, makes these available to the person via, in this embodiment a web based application 18 so that the person can then decide to follow the recommendation and purchase a pair of shoes which is indicated as being best suited to their particular detected parameters and thereby ensure that the selected shoes are most suitable for their purpose and the user. Thus, a subset of the available range of footwear may be generated and presented to the person for selection and purchase and so the person can make a more informed choice as to which of the shoes they are best suited to purchase.

FIG. 1b illustrates one embodiment of the smart phone application & database server which is a bespoke developed smartphone application and backend database server application which consist of a number of elements for user interaction and allowing connectivity of the collected data for onward processing.

Typically, the data processing means 12 includes a pre-processing SQL database which receives the consumer data from the smart phone 8 application 9 via the smartphone's Internet data connection, and the SQL pre-processing database then formats the person's data and pushes the person's data to the bespoke developed AI Algorithm program 14, which also resides on the server.

The AI Algorithm 14, when it receives the consumer data, undertakes a series of pre-processing steps, including data normalisation and dimensionality reduction. The person's data is then modelled and output with a landing pattern number and contact time and the AI output representative data group for that person is then be forwarded to the post-processing SQL database for data analysis and product matching and first the representative data group is used to update the user's data record and attempts to match the data profile for that person to the most suitable type(s) of running shoe in the product database.

Also, at any time, the person has the option to review their data record and profile and then review and refine the product (i.e. running shoe) database by one or more user specified filters such as manufacturer, colour, size, price, and drilldown to review the individual running shoe specifications.

One possible embodiment of the device and carrier for the same is illustrated in FIGS. 2a-f. The device 2 in the embodiment shown in FIGS. 2a-d and 2f is provided to be carried by a support sock 20, formed of neoprene or a similar material, to allow the same to wrap around at least part of the foot 22 and/or ankle 24. This allows the sock 20 to stretch around any size foot and the device 2 with the sensor or sensors is contained in a pocket 26 on the top face 28 of the sock 20. The carrier may also carry information and/or branding thereon and can be provided in different colours.

In an alternative embodiment shown in FIG. 2e the device 2 is provided to be worn on the foot 22 and ankle 24 via a carrier formed by straps 30. In one embodiment, the shape and size of the carrier can be selected to suit particular users and/or particular parameters which are required to be detected for the particular purpose of the footwear. The particular location or locations at which the device is worn can be selected to obtain the optimum position for the sensor or sensors 4 and in FIG. 2e it will be seen that these are spaced along the straps 30.

In each case, the device is worn and most typically die person will be asked to perform one or more predesignated tasks such that the required parameter measurements can be obtained in a uniform manner. FIG. 2f illustrates the carrier in the form of the sock 20 of FIGS. 2a-d being worn in use, with the accessible sensor device pocket 26 sewn into the foot bridge 28 of the support sock, to house the device 2 and the device 2 is removable from and insertable into the pocket 26.

In each embodiment the device 2 is provided to collect data which allows the analysis of parameters of the foot with which the device is being worn, and as a result, physical attributes of the person who is wearing the device at that time. The person may also be requested to enter other parameters into the system such as their gender, terrain on which the shoe is to be used, previous injuries etc.

In one embodiment the parameters which are determined using the data from the device 2 are the user's footstrike, gait and/or pronation. In one embodiment the foot strike parameter includes the foot strike at the heel, flat base of the foot and toes.

It should also be appreciated that the number and types of sensors 4 which are provided on the device are selected to suit particular purposes of use of the device. For example, the examples described and discussed in detail herein, relate to the provision and an analysis of data which allow particular running shoe types to be recommended, However the device can be adapted to include more, or different, sensors to provide alternative data, such as data relating to the health condition of the foot of the user, or more general health of the person and the use of the device in this manner is incorporated herein as part of the invention. In one embodiment the further or alterative sensors can include foot pressure pad sensors and the data may be used to create 3D imagery to provide a graphical representation of the condition of the foot.

In one embodiment the device includes a Printed Circuit Board (PCB) 32, an example of the front surface 34 of which is illustrated in FIG. 3a and which includes a power cell mounted to the rear of the same. A 3 axis accelerometer sensor 4 is also provided and this is illustrated graphically in FIG. 3b with the 3 axes X,Y,Z of measurement indicated with respect to the PCB 32. In one embodiment the accelerometer has a minimum 12-bit resolution, can withstand a minimum 14 grams of force and is capable of being polled at up to 60 Hz. The accelerometer allows the impact, and direction of impact of the foot 22 on a surface 36 to be determined.

Also provided is a 3 axis gyroscope sensor 4′ as illustrated graphically in FIG. 3c which shows the three axes X,Y,Z about which the measurements are generated as indicated by arrows 38,40,42 and which has a minimum of 16-bit resolution, a range of 250/2000 deg/s of rotation and is capable of bring polled at up to 60 Hz. The gyroscope provides data which allows the tilt and/or roll of the user's foot and/or up and down movement of the users toot to be determined.

The device also includes a thin film pressure sensor 4″ and one example is shown in FIG. 3d. The pad includes external faces, 44, 46 formed of a flexible material and sandwiched between these layers are first and second conductive material layers 48, 50, and intermediate those layers there are provided pressure sensitive layers 52, 54 and an adhesive layer 56 which keeps the respective layers in position as pressure placed onto the same in the direction of arrow 58.

The PCB also includes a Bluetooth communication module and a protective enclosure is provided to protect the device components in use.

In one embodiment the device interacts, in one embodiment using Bluetooth, with an app which is installed on the person's device such as a mobile phone which displays the parameter measurements obtained and sends them to the cloud based database.

In one embodiment the device can be worn on each of the user's feet in succession by removing the sock and device from one foot and then placing it on the other. Typically the subsequent data processing generates an “average” set of data based on the readings from each foot.

In operation, in one embodiment, the device is activated by a physical tap on the device which enables the Bluetooth module. The device will then bond with a previously connected smartphone or if a previous bond does not exist, the device will advertise for a connection and upon success, the device will pair and bond to a smartphone.

The smartphone will retrieve the unique device identification from the device and determine whether to accept the bond or reject the current bond. Upon acceptance, the device will save the bond for future use and add it to other accepted bonds from previous connections and the device will advertise for these bonds. If a smartphone is not found within 30s, the device will advertise for a new connection and the procedure for new connections is followed.

Typically the device will remain operational whilst a connection is in place hut after a predetermined time, such as for example 3 minutes, of inactivity (no movement or connection), the device will power off.

Typically the smartphone will configure the range for the accelerometer and the gyroscope of the device and shall configure the warm-up time for running, the running period and the poll rate i.e. number of measurements per second. Typically the longer the measurement period and the higher the poll rate then the more data there will be to store and the transfer time for the data will be longer when complete.

In order to commence a measurement period the smartphone will issue a start command and the device will pause for the warm-up period before polling the accelerometer and gyroscope sensors at the configured poll rate and the data collected for that period is then stored on memory of the smartphone. If a stop command is received at any time, the measurement will terminate and any measurements recorded will be made available. The data will be recorded into persistent memory and will therefore be available until the next measurement period commences.

Typically in order to read a measurement a status request is issued and the response contains the number of records and any error encountered. The number of records will indicate how many records will be in the measured data response message. Each record can contain up to 2 sensor measurements of accelerometer and gyroscope x,y,z values. Each sensor record is separated by 1/poll time, so a poll time of 50 Hz would have a 20 ms delay between measurements and 40 ms delay between records.

Typically, when the measurement data is streamed each record is designed to fit in one Bluetooth packet (with a payload of 20 Bytes).

In one embodiment the cloud based database will store details relating to a range of running shoes which are available at a particular time and typically the database will be updated at regular intervals. The data base will typically include technical details relating to the “type” and “suitability” of each of the running shoes and the data can be provided by the manufacturers and/or may be independently generated. The metadata about the product will typically include categories of shoes suitable for different type of runners, i.e. support/structured, cushioned/neutral, barefoot. To allow a standard product view of the store to be filtered by the customer not only on personal choice, such as any or any combination of, gender, price, size, colour, weight, style and/or heel drop, but specifically for foot type supplied from the wearable device. The Smartphone app, in one embodiment will have two interfaces, a first which will connect to the cloud database over the Internet enabling the presentation of shoes available via an approved retailer. The second interface will be an interface to the wearable sensor and as a result of the comparison of these two datasets, the range of available footwear can be filtered, with additional filters that can be set on the UI of the app to create a subset of best suited available footwear for purchase.

The present invention therefore provides a wireless motion device which takes a series of key parameter measurements of a foot. As an alternative to being strapped around the ankle of the foot the device can be clipped onto an ankle sock and in any embodiment worn for a period of time such as a minimum of one minute during which the person runs/jogs so that the device can calculate a dynamic and average measurement of key movement parameters such as foot strike, pronation and contact time.

The measurements are then transmitted to processing means which transmits the data to a database of training/running shoes including a variety of all the latest brands and models. Using the information that has been transmitted, the database then provides the customer with a ‘shortlist’ of suitable footwear across a range of different manufacturers/brands to consider, giving them reassurance that those particular shoes will be the most suitable for their particular foot and running style and this provides consumers with choice and confidence to buy footwear that is best suited to them.

The present invention therefore provides a wearable technology solution that allows the shoe purchaser, typically a runner to receive advice, in the convenience of their own home or another venue chosen by the purchaser, which is provided with reference to the analysis and understanding of that purchaser's specific running gait characteristics with regard to, for example, their foot strike, pronation and contact time on the surface.

1. A system for generating data representative of a person and using that data as criteria in a selection made for that person, said system comprising:
a device to be worn on a foot of the user, wherein said device includes sensing means including an accelerometer a gyroscope and a pressure sensing means to allow one or more parameters relating to that person's foot to be measured during one or more physical activities performed by the person and data relating to the one or more parameters is collected and transmitted via a transmission means to a data processing means to allow a representative data group to be generated for the said person and matching means compare the said representative data group to data stored in a memory which includes data representative of a range of footwear and indication means are controlled to indicate, from the result of the matching means, any of the said range of footwear which are selected to be suggested to be worn by the said user.

2. A system according to claim 1 wherein the processing means allows a recommendation of one or more items of footwear to be made for the said person based on the measured parameter of data.

3. A system according to claim 1 wherein the processing means refers to data relating to the range of footwear to compare the measured data with the same one determining which items of footwear are suitable.

4. (canceled)

5. A system according to claim 1 wherein the data which is generated by the device in relation to one or more parameters is stored and/or communicated to a database at a remote location which processes the data with reference range of available items of footwear which are available at that time and, if available, recommends one or more items of the footwear which are best suited to the persons feet as represented by the data.

6. (canceled)

7. A system according to claim 1 wherein the system informs the person if a specific manufacturer and model of a shoe is suitable for them based on a comparison of the parameter measurements for that person which had previously been obtained from the device and predefined data relating to characteristics of the particular shoes.

8. A system according to claim 1 wherein the parameter measurements are identified for both feet and data is provided for both feet.

9. A system for generating data representative of a person and using that data as criteria in a selection made for that person, said system including a device to be worn on a foot of the user, wherein said device includes sensing means to allow one or more parameters relating to that person to be measured during one or more physical activities performed by the person and data relating to the one or more parameters is collected and transmitted via a transmission means to a data processing means to allow a representative data group to be generated for the said person and matching means compare the said representative data group to data stored in a memory and indication means are controlled to indicate the result of the matching means.

10. A system according to claim 9 wherein the sensing means include an accelerometer, a gyroscope and a pressure sensing means.

11. A system according to claim 9 wherein the at least one parameter is linked to the person's foot and the said data stored in memory with which the representative data group is compared is data relating to a range of footwear which is available to be purchased, historical data relating to one or more elements relating to that persons health, historical data relating to one or more elements relating to expected health values, data relating to other parameters which is input and used in the processing of the data for that person, or any combination thereof.

12-15. (canceled)

16. A system in accordance with claim 15 wherein the said further data includes any or any combination of height, weight and/or age of the person and/or type of terrain in which footwear is to be worn and/or other criteria relating to the footwear such as price and/or colour.

17. A device to be mounted in position on or adjacent to a foot of a person, wherein said device includes sensing means including an accelerometer a gyroscope and a pressure sensor to allow one or more parameters relating to that person's foot to be measured during one or more physical activities performed by the person and data relating to the one or more parameters is collected and transmitted via a transmission means from the device for subsequent processing wherein the said sensing means are located on a carrier to be placed and retained on the foot.

18. A device according to claim 17 wherein the device is provided to be located when in use in a carrier which includes engagement means, or is formed in a manner, so as to allow engagement of the carrier with the foot and to maintain the device in a required position with respect to the foot during the performance of the one or more activities.

19. A device according to claim 17 wherein the carrier is a sock or part of a sock, a bracelet, anklet or like which fits around the ankle of the wearer and/or around part of the foot of the person so as to be located to provide required data protection points on the users foot.

20. A device according to claim 17 wherein the device collects measurement data linked to at least four biodynamic parameters to the foot when the customer is asked to perform an exercise.

21. A method for the analysis of one or more parameters relating to a person's foot, said method comprising the steps of providing and using a combination of sensing means provided as part of a device and locating the device on or adjacent to the foot whilst the person performs at least a walking and/or running action for a period of time and/or for a particular distance, collecting data from the sensing means which is created during the movement of the person's foot, transmitting the data from the device to data processing means and processing the data using one or a series of algorithms to output a representative data group related to that person to a post processing database and performing a matching exercise with respect to a database of data relating to a range of shoes and upon the results of the match selecting one or more pairs of shoes from the range which are suggested to the person as being best suited for them to wear.

22. A method according to claim 21 wherein each of the available pairs of shoes are allocated specific identity data so as to allow the same to be matched, or not, with the representative data group for a person.

23. A method according to claim 21 wherein the results are presented to the person via an interface which can be viewed on a smart phone or within a PC/Mac web browser interface and the data is collected from the device via a wireless connection, to a transmitting device, which then transmits the collected data to a data processing means at which the algorithms are processed.

24. (canceled)

25. A method according to claim 21 wherein the data processing means and/or post processing means are a facility which is remote from the location of use of the device and receive and process collected data from a number of devices.

26. (canceled)

27. A device according to claim 17 wherein the sensors are contained in a pocket on the top face of the carrier when worn.

28. A device according to claim 27 wherein the pocket is provided on the foot bridge of the carrier
